(19) United States
(12) Patent Application Publication
(10) Pub. No.: US 2016/0360968 A1
(43) Pub. Date: Dec. 15, 2016

(54) FNIRS SYSTEM AND SENSOR ASSEMBLY

(71) Applicant: The Research Foundation for The State University of New York, Albany, NY (US)

(72) Inventor: David W. Bernat, New York, NY (US)

(73) Assignee: The Research Foundation for The State University o f New York, Albany, NY (US)

(21) Appl. No.: 15/098,677

(22) Filed: Apr. 14, 2016

Related U.S. Application Data
(60) Provisional application No. 62/147,357, filed on Apr. 14, 2015.

(51) Int. Cl. A61B 5/00; A61B 5/0478; G01N 21/359

(52) U.S. Cl. CPC A61B 5/0075; G01N 21/359; A61B 5/0042; A61B 5/6803; A61B 5/0478; G01N 2201/061; A61B 2562/0238; A61B 2562/066; A61B 2562/046

(57) ABSTRACT

Disclosed are sensor assemblies and a Functional Near-Infrared Spectroscopy (fNIRS) system. A sensor assembly includes at least one a motor configured to vibrate a sensing optode at a first frequency. A system includes a plurality of sensor assemblies and a controller for processing signals from the sensor assemblies.

### CROSS REFERENCE TO RELATED APPLICATIONS

The present application claims benefit of U.S. Provisional Application No. 62/147,357, filed on Apr. 14, 2015, the contents of which are incorporated herein by reference.

### FIELD OF THE DISCLOSURE

This disclosure relates to fNIRS imaging. More particularly, this disclosure relates to a system for imaging a subject and a sensor assembly or sensor mount.

### BACKGROUND

Functional Near-Infrared Spectroscopy (fNIRS) is used for imaging a patient including for neuroimaging. Light emitters and detectors are placed in the vicinity of an area of interest, e.g., the subject's skull. This placement allows for recording activity due to reflected light. The observation of internal absorption (due to blood volume and concentration changes) is measured using optode systems with light emitter and detectors. The desired signals, e.g., internal, however are impacted by changes in contact with the external surface and changes in the surface. For example, the measurement is impacted by movement, scalp skin deformation, hair absorption, among other factors. This impact is in the form of noise which cannot be directly calibrated out and cause unwanted time-varying derivations.

### SUMMARY

Disclosed is a Functional Near-Infrared Spectroscopy (fNIRS) system. The system comprises at least four sensor assemblies. Each sensor assembly has at least one optode system. Each of the at least one optode systems includes a light source and a light detector. The light source emits light at a preset duty cycle.

The light source is configured to emit light. The light detector in a sensor assembly is capable of detecting emitted light from the light sources from other sensor assemblies.

A light source in one sensor assembly and a light detector in another sensor assembly form a source-detector pair. The light detector in the source-detector pair receives emitted light from the light source in the source-detector pair.

The at least four sensor assemblies form at least twelve source-detector pairs, where the light detectors produce at least twelve signals, respectively, representing the detected light detected by the light detector in the source-detector pair.

The sensor assemblies are spaced apart from one another.

The system further comprises a controller electrically coupled to each of the at least four sensor assemblies. The controller is configured to: control the light source in each of the at least four sensor assemblies, receive respective signals from each light detector, select at least four signals of the at least twelve signals for collective processing, and process the selected at least four signals of the at least twelve signals to determine absorption. The at least four signals are received from source-detector pairs forming a closed loop arrangement.

Also disclosed is a sensor assembly which comprises a tubular member. The tubular member has a distal portion and a proximal portion. The tubular member also has at least one axial opening extending the length of the tubular member from the proximal portion to the distal portion forming a shaft configured to receive at least a portion of an optode. The at least one axial opening has a diameter of a preset size. The tubular member has an external surface. The sensor assembly further comprises a first support and a second support mounted to the external surface of the tubular member. The sensor assembly further comprises an urging member support opposing a proximal facing portion of the tubular member when the optode is inserted in the shaft. The sensor assembly further comprises a first set of urging members including a first urging member and a second urging member. The first urging member extends between the urging member support and the first support. The second urging member extends between the urging member support and the second support. The first and second urging members are configured to, when the optode is inserted in the shaft, urge the optode to a target area. The sensor assembly further comprises a motor configured to, when the optode is inserted in the shaft, vibrate the optode at a first frequency.

### BRIEF DESCRIPTION OF THE FIGURES

FIG. 1 illustrates a diagram of a sensor assembly in accordance with aspects of the disclosure;

FIG. 2 illustrates a diagram of a second sensor assembly in accordance with aspects of the disclosure;

FIG. 3 illustrates a diagram of a third sensor assembly in accordance with aspects of the disclosure;

FIG. 4 illustrates two Source-Detector Pairs from two Sensor Assemblies in accordance with aspects of the disclosure;

FIG. 5 illustrates an example of a sensor assembly configuration showing Source-Detector Pairs in accordance with aspects of the disclosure;

FIG. 6 illustrates an example of a sensor assembly configuration showing four signals from Source-Detector Pairs which form a closed loop in accordance with aspects of the disclosure;

FIG. 7 illustrates a block diagram of a fNIRS system in accordance with aspects of the disclosure;

FIG. 8 illustrates a flow chart for a method of imaging in accordance with aspects of the disclosure;

FIG. 9A illustrates an example of a four sensor assembly arrangement around an EEG Electrode of a 10-20 system in accordance with aspects of the disclosure;

FIG. 9B illustrates an example of two sensor assembly configurations, each showing of four individual signals from Source-Detector Pairs for combining in accordance with aspects of the disclosure;

FIG. 10 illustrates an example of fNIRS Sensor Assembly Configuration within a 10-20 international System;

FIG. 11 illustrates an example of fNIRS Sensor Assembly configuration having different imaging depths;

FIG. 12 illustrates experimental results depicting signals from detectors in two Source-Detector Pairs; and

FIG. 13 illustrates experimental results depicting attenuations determined from signals from detectors in four Source-Detector Pairs forming a closed loop and the combined attenuation determined in accordance with aspects of the disclosure.

### DETAIL DESCRIPTION

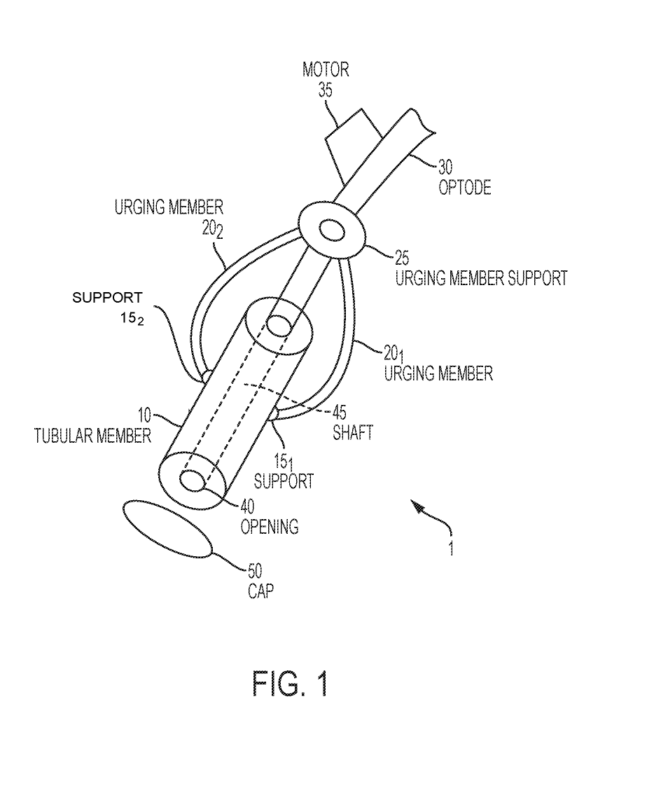

FIG. 1 illustrates a diagram of a Sensor Assembly 1 or Sensor Mount in accordance with aspects of the disclosure for use in imaging a patient. The Sensor Assembly 1 includes a tubular member 10. The tubular member 10 can be made of rubber. For example, the tubular member 10 can be a rubber stopper. The tubular member 10 has a proximal portion and a distal portion. As depicted in FIG. 1, the distal portion faces a cap 50. The cap 50 will be described later in detail. As depicted in FIG. 1, the tubular member 10 includes one opening 40. The opening 40 extends the entire length of the tubular member 10 in the axial direction (e.g., longitudinal axis). The opening 40 creates a shaft 45 in the tubular member 10. The shaft 45 is dimensioned to hold an optode 30. The diameter of the opening and thus the shaft can be equal to the diameter of the optode 30 such that the optode snugly fits within the shaft 45. The axial length of the tubular member 10, e.g., distance between the distal end and the proximate end is small enough to fit within a slot in a standard 10-20 International EEG cap.

Supports 15₁ and 15₂ (collective supports 15) are mounted to the outside of the tubular member, e.g., the external surface. In an aspect of the disclosure, the supports 15 are mounted on opposing sides of the tubular member 10. The supports 15 can be located symmetrically on opposite sides. The supports 15 can be a hook mounted on the side of the tubular member 10. The supports 15 are an attachment point for the urging members (collectives urging members 20).

The Sensor Assembly 1 also includes an urging member support 25. The urging member support 25 is located on a predetermined position on the optode 30. The urging member support 25 can be a small washer with a opening such that the optode 30 passes therethrough. One end of an urging member 20 is attached to the urging member support 25 and the other end of the urging member 20 is attached to one of the supports 15. For example, urging member 20₁ is attached to the support 15₁ on one end and the urging member support 25 on the other end. Similarly, urging member 20₂ is attached to support 15₂ on one end and the urging member 25 on the other end.

The urging member 20 can be an elastic band. Alternatively, the urging member 20 can be a spring. The urging member provides force on the optode to ensure that the optode is fully inserted into the tubular member 10 and remains in contact (direct or indirect) with the target site. The length and tension of the urging member 20 is selected such that the distal end of the optode is flush with the distal end of the tubular member 10. Additionally, the position of the urging member support 25 is set such that the position in combination with the urging member 20 cause the distal end of the optode 30 to be flush with the distal end of the tubular member 10.

Separation of the optode 30 from the target site, such as the scalp, will produce destructive articles and is a major factor of noise.

The Sensor Assembly 1 also includes a motor 35. The motor 35 is coupled to the optode 30. The motor 35 is a vibration motor. The motor 35 is set to vibrate the optode 30 at a frequency greater than a data sampling rate. The data sampling rate will be described in detail later. The vibration of the optode 30 at the set frequency improves the coupling coefficient of the optode 30 and the target site and will lead to a more accurate measurement, such as the measurement of the true cortical absorption. For example, the motor 35 can be a 1.5 V or 3 V motor.

A coupling coefficient between the optode 30 and the target site is dependent on the percentage of light leaving the target site that is channeled through a fiber cable to the detector. The coupling coefficient is highly sensitive to the angle between the optode 30 and the target site. It is a maximum when the optode 30 is directly perpendicular to the target site.

The combination of the motor 35 vibrating the optode 30 and the force exerted by the urging member 20 on the optode 30 controls contact with the site to provide a random and rapid orientation changes with respect to tilt angle of the optode 30 and target site in a single exposure. Thus, a signal output by the detector, e.g., a single exposure, effectively is an average of the rapidly changing orientations.

The Sensor Assembly 1 further includes a cap 50. In an aspect of the disclosure, the cap 50 can be detachable from the tubular member 10. The cap 50 includes a flat surface. The cap 50 is configured to orient the tubular member 10 perpendicular to the target site. In another aspect of the disclosure, the cap 50 can be affixed to the tubular member 10 without being detachable.

In FIG. 1, the cap 50 is shown as being separate from the tubular member 10 for illustrative purposes only, in operation, the cap 50 is flush with the distal end of the tubular member 10. In an aspect of the disclosure, the cap 50 is transparent to near infrared light.

When the Sensor Assembly 1 depicted in FIG. 1 is used with a single optode 30, the optode acts as both a light source and a light detector. During a portion of a duty cycle, the optode 30 acts as the light source, whereas in the remaining portion of the duty cycle, the optode acts as the light detector.

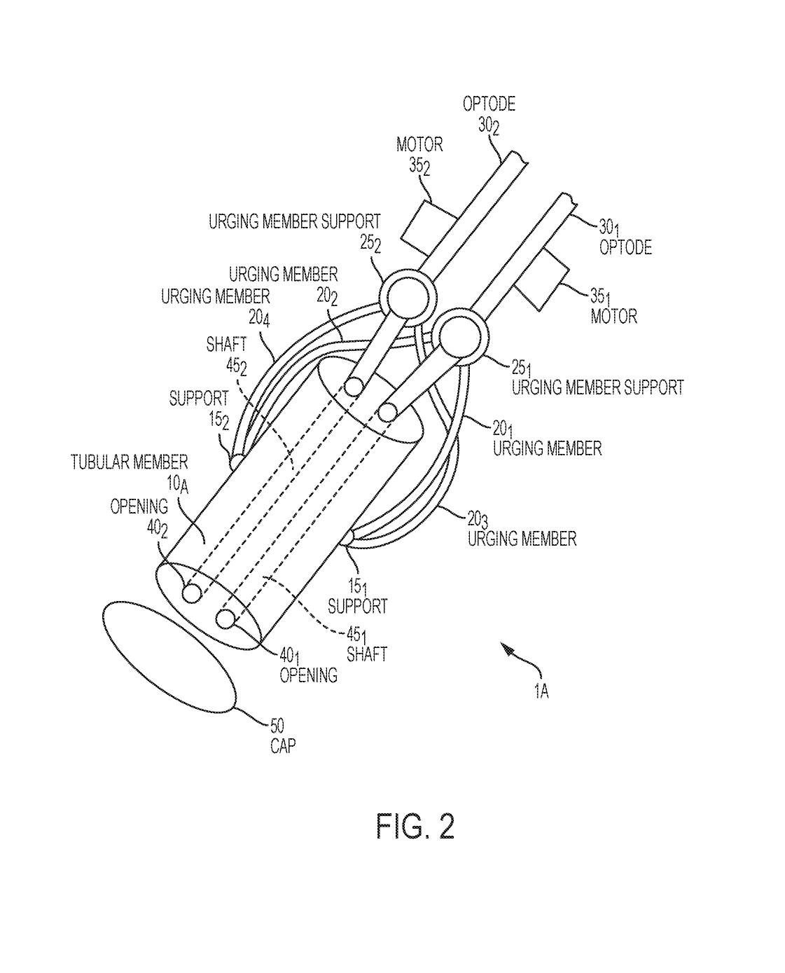

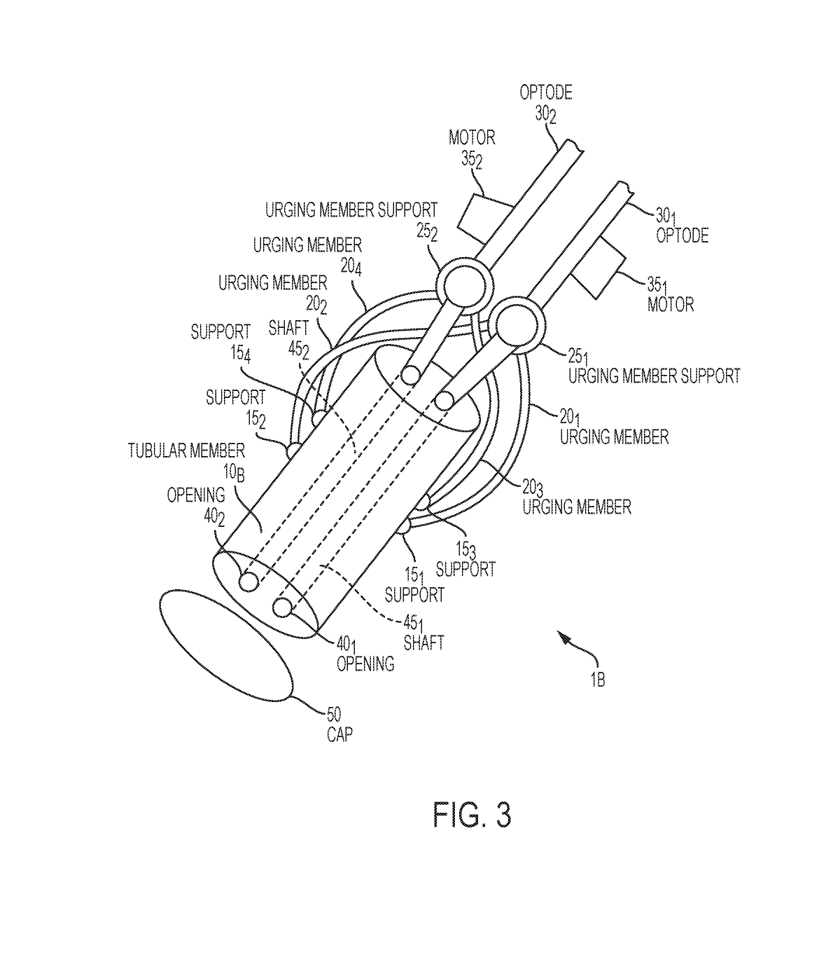

In another aspect of the disclosure, a pair of optodes can be included in a sensor assembly. FIGS. 2 and 3 illustrate examples of Sensor Assemblies 1A and 1B for housing two optodes. Like reference numbers are used for similar components. Many of the components of the Sensor Assembly 1A are similar to the components described above and will not be described again in detail.

The tubular member 10A includes two openings 40₁ and 40₂. The openings extend the entire length of the tubular member 10A in the axial direction (longitudinal axis). The openings 40₁ and 40₂ create respective shafts 45₁ and 45₂ in the tubular member 10A. The shafts 45₁ and 45₂ are dimensioned to hold respective optodes, e.g., 30₁ and 30₂. The diameter of the openings and thus the shafts can be equal to the diameter of the optodes 30 such that the optodes snugly fits within the shafts. As with the tubular member 10 depicted in FIG. 1, the axial length of the tubular member 10A, e.g., distance between the distal end and the proximate end is small enough to fit within a slot in a standard 10-20 International EEG cap. The distance between the openings is small. In particular, the distance between the openings is smaller than the distance between different sensor assemblies.

The Sensor Assembly 1A also includes two urging member supports 25₁ and 25₂. Each urging member support is located on a predetermined position on a respective optode 30. Each urging member support has an opening such that the optode 30 passes therethrough.

The Sensor Assembly 1A also includes two sets of urging members (20₁ and 20₂ and 20₃ and 20₄). One set of urging members 20₁ and 20₂ is attached between the urging member support 25₁ and support 15₁ and 15₂. The other set of urging members 20₃ and 20₄ is attached between the urging member support 25₂ and support 15₁ and 15₂.

The length and tension of the sets of urging members are selected such that the distal end of the respective optodes are flush with the distal end of the tubular member 10A. Additionally, the position of the urging member supports 25₁ and 25₂ are set such that the position in combination with the sets of urging members cause the distal end of the respective optode to be flush with the distal end of the tubular member 10A.

The Sensor Assembly 1A also includes two motors 35₁ and 35₂ (Collectively references as motors 35). The motors are coupled to a respective optode, e.g. motor 35₁ is coupled to optode 30₁ and motor 35₂ is coupled to optode 30₂. The motors 35 are a vibration motor. The motors 35 are set to vibrate the respective optode at a frequency greater than a data sampling rate.

FIG. 3 illustrates another example of a Sensor Assembly 1B in accordance with aspects of the disclosure for two optodes. The difference between the Sensor Assembly 1B and the Sensor Assembly 1A is that the tubular member 10B in Sensor Assembly 1B (FIG. 3) has a second set of supports 15₃ and 15₄ mounted to the external surface of the tubular member 10B. Thus, unlike the Sensor Assembly 1A where the sets of urging members are attached to the same supports (15₁ and 15₂), in the Sensor Assembly 1B, each set of urging members are attached to different supports. The first set of urging members (20₁ and 20₂) are attached to support (15₁ and 15₂) whereas the second set of urging members (20₃ and 20₄) are attached to support (15₃ and 15₄).

The remaining configuration of Sensor Assembly 1B is the same as Sensor Assembly 1A and will not be described again.

In operation, when a pair of optodes is mounted in a sensor assembly, e.g., 1A and 1B, one of the optodes acts as a light source and a second optode acts as a light detector. Pairs of optodes within different sensor assemblies form pairs of sources (S) and detectors (D) (referenced as a Source-Detector Pair).

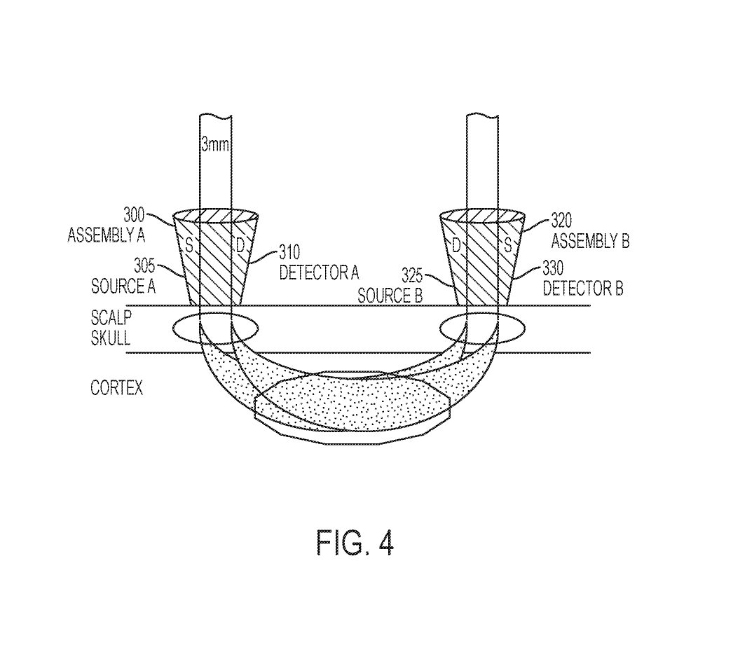

FIG. 4 illustrates two Sensor Assemblies and two Source-Detector Pairs. For purpose of description, the sensor assemblies in FIG. 4 will be referenced as Assembly A 300 and Assembly B 320. The optodes in Assembly A 300 will be referenced as Source A 305 and Detector A 310. The optodes in Assembly B 320 will be references as Source B 325 and Detector B 330.

The two Source-Detector Pairs are Source A 305—Detector B 330 and Source B 325—Detector A 310. Each Source-Detector Pair measures the attenuation or absorption of infrared light. The attenuated light received by the detector reflects the absorption of the light between the Source and the Detector.

In the example depicted in FIG. 4, the Sensor Assembly A 300 in combination with Sensor Assembly B 320 are capable of measuring absorption in the scalp and skull as well as the absorption within a cortex (represented by the three circles in the figure).

The ratio of received light to emitted light is the absorption (attenuation) of the light due to both the scalp and skull and cortex.

Φ_{AB}=G_{A}G_{B}S_{AB}. Equation (1)

Φ_{AB} is the attenuation=Amplitude of received light signal/Amplitude of the emitted light signal.

G_{A} is the attenuation or absorption by the scalp and skull at the point of contact where Sensor Assembly A 300 is placed.

G_{B} is the attenuation or absorption by the scalp and skull at the point of contact where Sensor Assembly B 320 is placed.

S_{AB} is the attenuation or absorption by the cortex between the points of contact.

Further log

Φ_{AB}=log G_{A}+log G_{B}+log S_{AB}. Equation (2)

As noted above, the distance between optodes with the same sensor assembly is smaller than the distance between sensor assemblies. As depicted in FIG. 4, the distance between optodes within the same assembly, e.g., as Source A 305 and Detector A 310, is 3 mm. The distance between Assembly A 300 and Assembly B 320 is approximately 3-4 cm. Because the spacing between optodes in each sensor assembly is less than the spacing between the sensor assemblies, the two Source-Detector Pairs measure approximately the same absorption region (represented by the two banana-shaped paths being slightly offset).

Since the absorption region is similar, signals from the respective detectors are nearly identical.

Φ_{AB}=Φ_{BA} Equation (3)

Deviations between the signals are indicative of systematic error and potential misalignment between the two Source-Detector Pairs.

In an aspect of the disclosure, at least four sensor assemblies are used for imaging. Each sensor assembly can include either one optode acting as both the Source and Detector or two optodes, one acting as the Source and the other acting as the Detector.

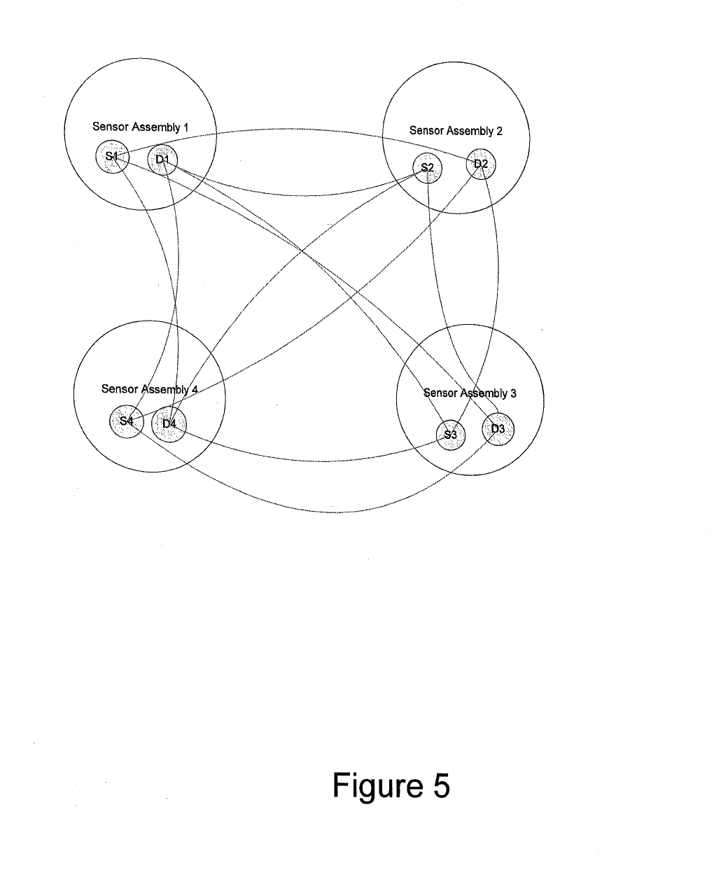

FIG. 5 illustrates four sensor assemblies (Sensor Assembly 1-4). Each sensor assembly includes a Source (S) and a Detector (D). Light emitted from a Source (S) can be detected by the detectors within each other Sensor Assembly. For example, light emitted by Source S1 can be detected by detectors D2, D3 and D4. Similarly, light emitted by Source S2 can be detected by detectors D1, D3 and D4. Light emitted by Source S3 can be detected by detectors D1, D2 and D4. Light emitted by Source 4 can be detected by detectors D1, D2 and D3. In FIG. 5, the lines between the sources and each detector represent the light for illustrative purposes only. The lines are separated so that they can be individually identified.

Each time Sources S1-S4 emit light (one cycle), they produce twelve detection results (detected signal).

In the context of the description, Source-Detector Pair refers to a detector detecting light emitted from a specific source. In the example shown in FIG. 5, there are twelve Source-Detector Pairs: S1-D2, S1-D3, S1-D4, S2-D1, S2-D3, S2-D4, S3-D1, S3-D2, S3-D4, S4-D1, S4-D2, and S4-D3.

The signals can be combined to provide additional information for the imaging such as an attenuation or absorption.

In accordance with aspects of the disclosure, detection signals from at least four of the source-detector pairs are combined to eliminate absorption due to the scalp and skull. Any even number of detection signals greater than four can be used if forming a closed loop. For purposes of the description a closed loop means that the Sensor Assembly that includes the source of the first source-detector pair used in the combination is the same Sensor Assembly that includes the detector of the last source-detector pair used in the combination.

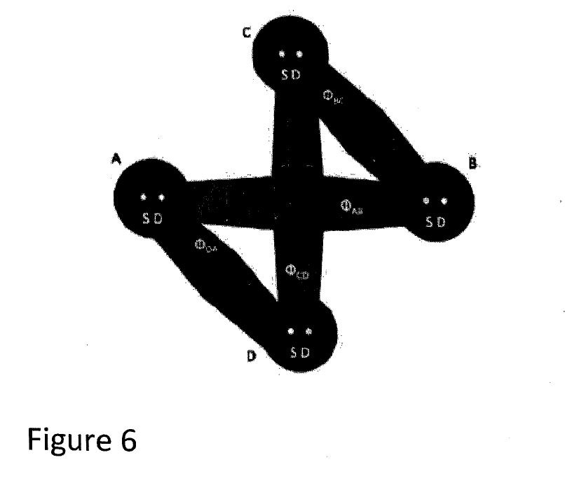

FIG. 6 illustrates an example of a four sensor assembly configuration (Sensor Assembly A-D). Each sensor assembly has a respective source S and detector D. Using the configuration depicted in FIG. 6, the detected outputs from four Source-Detector Pairs can be used to determine the cortical absorption. The arrows in the figure show an example of a closed loop.

As noted above, Φ is the attenuation or total absorption.

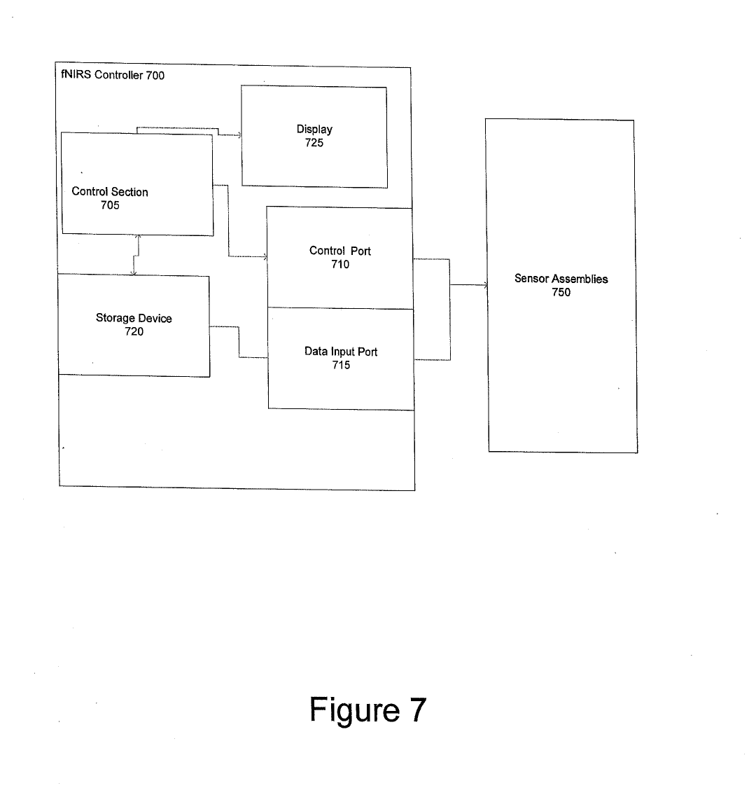

FIG. 7 illustrates a block diagram of a fNIRS system 760 in accordance with aspects of the disclosure. The fNIRS system 760 includes an fNIRS Controller 700 and Sensor Assemblies 750. The Sensor Assemblies 750 have been described above. The fNIRS Controller 700 controls the optodes in the Sensor Assemblies 750 using the Control Section 705. Additionally, the Control Section 705 is configured to calculate the attenuation/absorption based on the detected signals received from the Sensor Assemblies 750 via the Data Input port 715. The received signals are stored in a Storage Device 720 for processing. The optodes are controlled by the Control Section 705 via the Control Port 710.

In one aspect of the disclosure, the Control Section 705 is configured to control the Sources (optodes) within each Sensor Assembly to simultaneously emit light. In order for the detectors to distinguish the light emitted from each Source, the emitted light includes an embedded high frequency signal. The high frequency signal can be embedded into the emitted light by modulation. The light is emitted for a preset period of time and subsequently turned off by the Control Section 705, e.g., duty cycle. The emission of light can be repeated.

In another aspect of the disclosure, the Control Section 705 is configured to sequentially control the Sources (optodes) with each Sensor Assembly to emit light. For example, the Control Section 705 controls Source S1 to emit light. The light is detected by D2-D4. Then, the Control Section 705 controls Source S1 to stop emitting light. Subsequently, the Control Section controls Source S2 to emit light. The light is detected by D1, D3 and D4. Then, the Control Section 705 controls Source S2 to stop emitting light. The sequential control continues until all of the sources, e.g., Source S1-S4, emit light. Once all sources have emitted light and the detectors detect the same, the absorption/attenuation can be determined for the cycle.

The Control Section 705 controls the Display 725 to display the determined absorption/attenuation as in image.

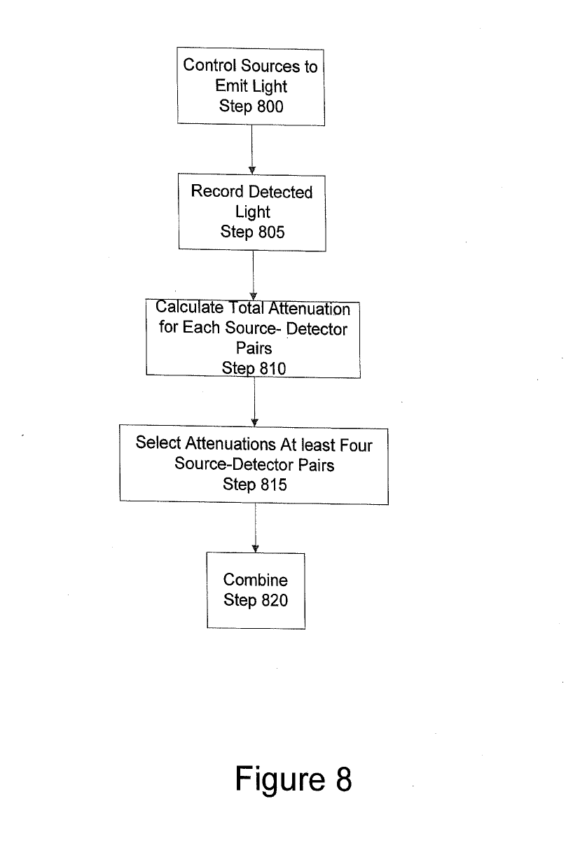

FIG. 8 illustrates a flow chart for a method of imaging in accordance with aspects of the disclosure. At Step 800, the Control Section 705 controls each Source S in the Sensor Assemblies to emit light. As described earlier, the Sources S can either be controlled to simultaneously emit light or sequentially emit light. The emitted light is detected by corresponding detector in Source-Detector Pairs. The detected emitted light is received via the Data Input Port 715 by the fNIRS Controller 700 and recorded in the Storage Device 720 (at step 805).

At Step 810, the Control Section 705 calculates a total attenuation for each Source-Detector Pair based on the detected emitted lights (from the Source-Detector Pairs). The phrase “total attenuation” refers to both the desired measured attenuation and noise. Since the Control Section 705 controls the Sources to emit the light, the Control Section 705 knows the magnitude of the emitted light. This magnitude is compared with the magnitude of the detected emitted light. The total attenuation for each Source Detector-Pair is calculated by

Φ=Amplitude of received light signal/Amplitude of the emitted light signal.

In an aspect of the disclosure, the amplitude of the emitted signal by each Source is the same.

The calculation is repeated for each Source-Detector Pair. For the configuration depicted in FIG. 5, 12 separate calculations are performed by the Control Section 705.

The calculated Φs are stored in the Storage Device 720.

At step 815, the Control Section 705 selected specific total attenuations for further processing. The selection is based on the desired absorption measurement or determination. At least four signals (attenuations) are selected. In an aspect of the disclosure, the number of selected signals (attenuations) is an even number. Additionally, the signals (attenuations) are selected for a closed loop.

FIG. 6 shows an example of four selected signals (attenuations). In FIG. 6, the Sensor Assemblies are identified as A-D.

Φ_{AB} is the signal (attenuation) from the Source S_{A} to Detector D_{B}. Φ_{BC} is the signal (attenuation) from the Source S_{B} to Detector D_{C}. Φ_{CD} is the signal (attenuation) from the Source S_{C} to Detector D_{D}. Φ_{DA} is the signal (attenuation) from the Source S_{D} to Detector D_{A}. In FIG. 6, four Source-Detector Pairs were selected.

For the square arrangement depicted in FIG. 6, the four selected signals (attenuations) are used to determine the cortex absorption (attenuation) at the center of the four Sensor Assemblies.

At step 820, the selected attenuations are combined to determine the cortical absorption. The determined cortical absorption does not include the absorption due to the scalp and skull.

The combination includes selectively adding and subtracting certain attenuations to eliminate the absorption due to the scalp and skull. For the example depicted in FIG. 6,

log Φ_{AB}=log G_{A}+log G_{B}+log S_{AB}.

Similarly,

log Φ_{BC}=log G_{B}+log G_{C}+log S_{BC}.

log Φ_{CD}=log G_{C}+log G_{D}+log S_{CD}.

log Φ_{DA}=log G_{D}+log G_{A}+log S_{DA}

For the configuration depicted in FIG. 6, the attenuation (absorption) by the scalp and skull can be eliminated by added log Φ_{AB} and log Φ_{CD} and subtracting log Φ_{BC} and log Φ_{DA}

log Φ_{AB}−log Φ_{BC}+log Φ_{CD}−log Φ_{DA}=log S_{AB}−log S_{BC}+log S_{CD}−log S_{DA} Equation (4)

FIG. 6 is presented by way of an example. Other geometry for the locations of the Sensor Assemblies can be used. Additionally, different signals (attenuations) can be selected for the combination by adding or subtracting the signals as long as a closed loop is created for the same geometric configuration.

In another aspect of the disclosure, the Sensor Assembly can be incorporated in a standard 10-20 International EEG Cap. Therefore, fNIRS imaging in accordance with aspects of the disclosure can be combined with EEG sensors. The Sensor Assemblies can be located within a 10-20 International EEG Cap such that the location of the fNIRS imaging and the determined absorption is co-located with the EEG (or any other point of contact sensor).

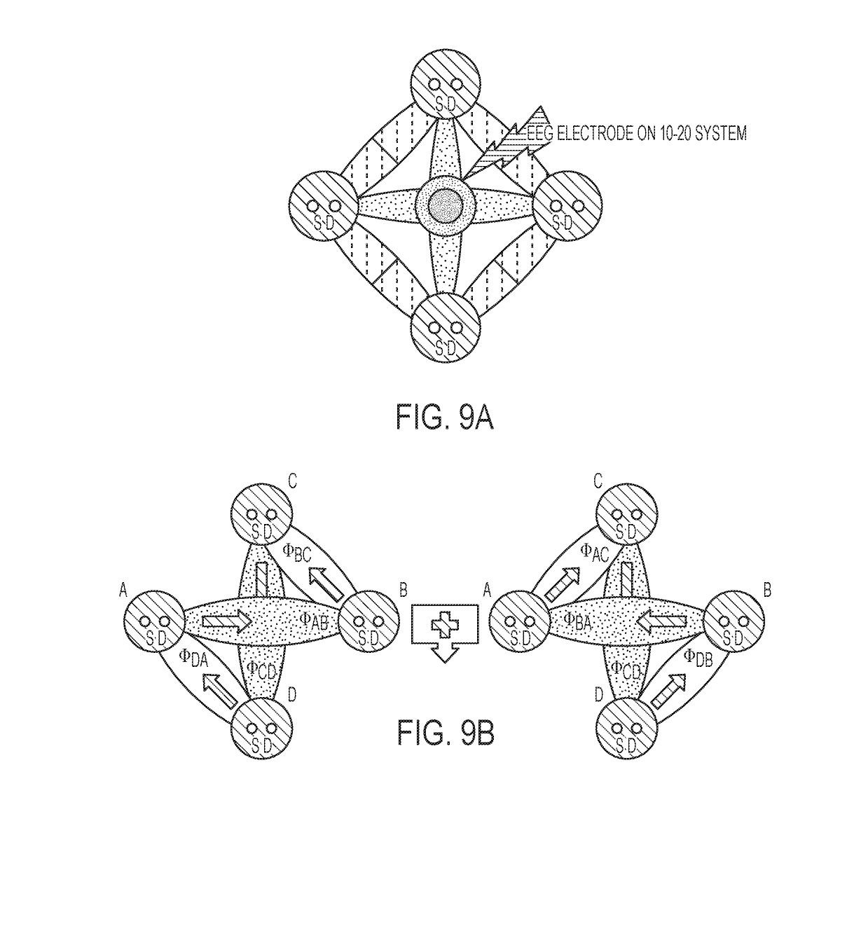

FIG. 9A illustrates an example of a four Sensor Assembly arrangement around an EEG Electrode of a 10-20 system. As depicted, the EEG Electrode is located at the center of the four Sensor Assembly arrangement. The four Sensor Assemblies are the same as depicted in the example from FIG. 6. Each has a Source S and a Detector D. This configuration allows for multimodal measurement of fNIRS and EEG with matching topological structures.

In the example depicted in FIG. 9A, signals (attenuations) from eight Source-Detector Pairs are combined. FIG. 9B shows the eight individual signals from the Source-Detector Pairs. Four signals in the left side of the figure and four signals on the right. The four from each are combined to generate the eight Source-Detector Pairs.

The signals (attenuations) from the above eight Source-Detector Pairs can be used to determine the difference between an increased absorption within the square and a decreased absorption around the edge.

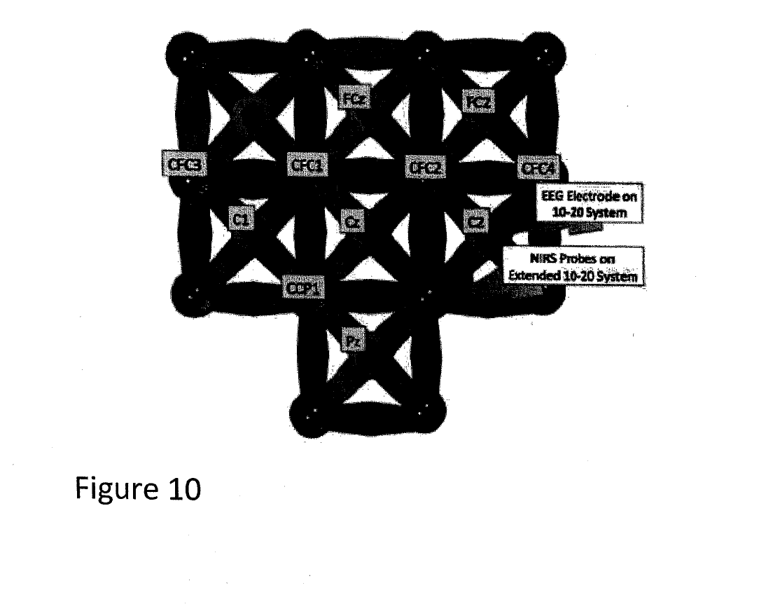

FIG. 10 illustrates an example of fNIRS Sensor Assemblies within the 10-20 international System.

In another aspect of the disclosure, the fNIRS Sensor Assemblies can be used in an ad-hoc measurement system.

The distance between the Sensor Assemblies is proportional to the depth of the imaging. The larger the distance between Sensor Assemblies, the imaging is deeper. The depth scales approximately linear with the Sensor Assembly separation.

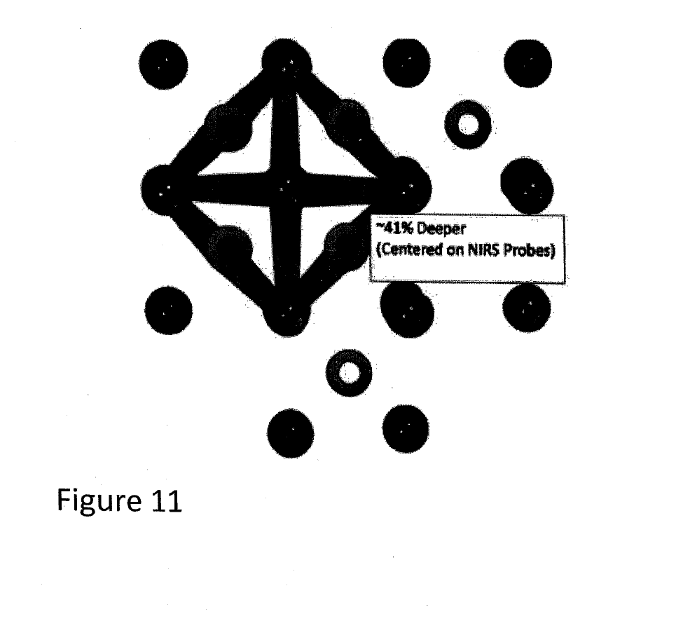

FIG. 11 illustrates an example of fNIRS Sensor Assembly configuration having different levels of imaging, thereby enabling mutli-slice topological imaging. For example, the four Sensor Assemblies showing emitted light have a 41% deeper imaging than the other fNIRS Sensor Assemblies depicted in FIG. 11.

In one aspect of the disclosure, the imaging system and method described herein uses the Sensor Assembly 1, Sensor Assembly 1A or the Sensor Assembly 1B described in FIGS. 1-3. In another aspect of the disclosure, the imaging system and method described herein can use another sensor assembly having one or more optodes (Source/Detector).

The system and method described herein can be used for any fNIRS imaging, such as breast and tissue imaging and is not limited to the cortex.

### Experimental Result

An experiment was conducted using six fNIRS Sensor Assemblies having optodes (Source and Detectors). The fNIRS Sensor Assemblies were installed in a standard swim cap. The six fNIRS Sensor Assemblies form 22 Source-Detector Pairs. The subject conducted a series of simply motor actions and imagery (e.g., absorption determined) interspersed with brief periods of rest.

Data from two Source-Detector Pairs were used to confirm that the imaging (e.g., absorption determined) is nearly identical when the Source-Detector Pairs are arranged as depicted in FIG. 4. The sampling rate was 4.3 Hz.

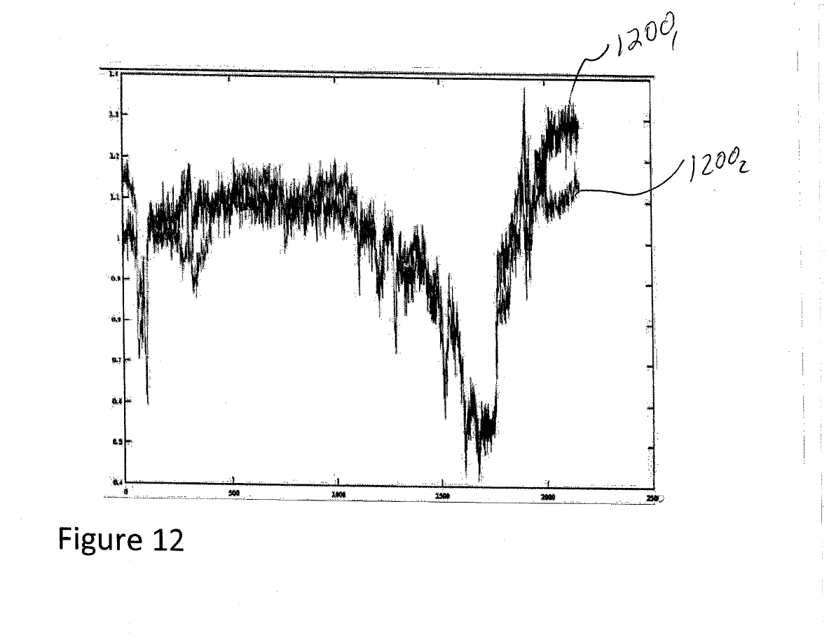

FIG. 12 depicts the data from the two Source-Detector Pairs 1200₁ and 1200₂. The x-axis is the measurement number. The y-axis is the detected light at the Detectors within the Source-Detector Pairs (after scaling). FIG. 12 shows that the detected values are nearly identical on a second and minute time scale and captures the relevant neurovascular and physiological trends.

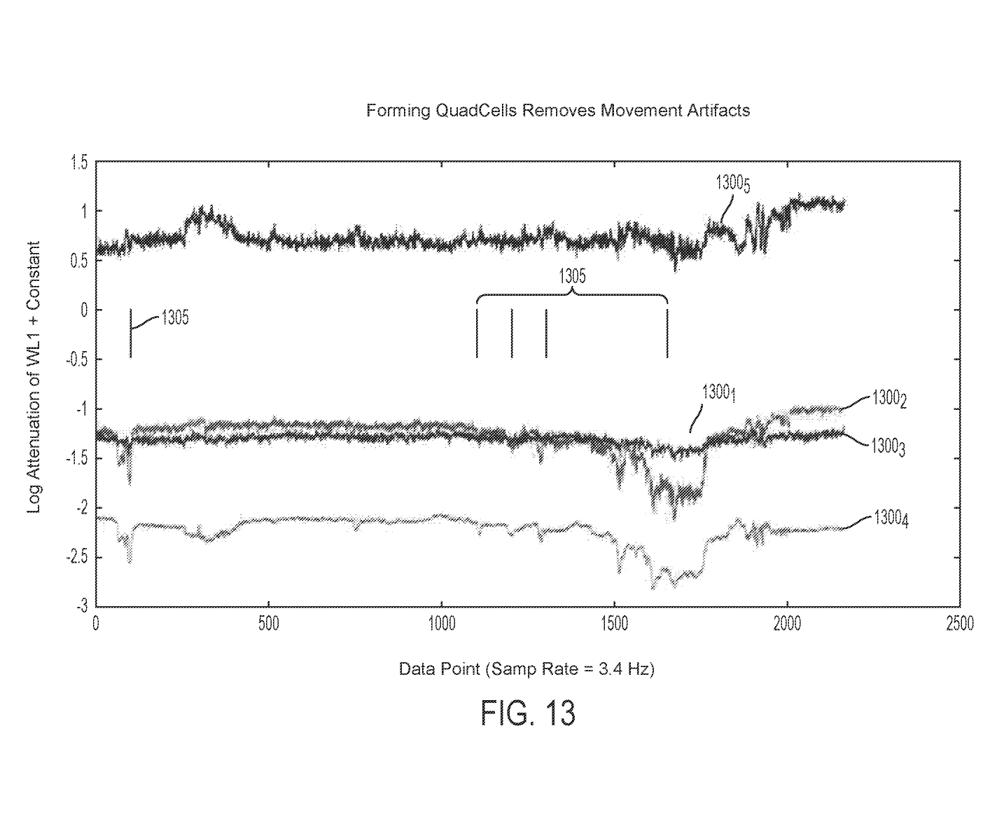

As disclosed herein any four signals (attenuations) from Source-Detector Pairs that form a closed loop can be combined. FIG. 13 depicts the results of the individual signals and a combined four signal value.

Traces 1300₁-1300₄ represent the log of the calculated attenuations from the four Source-Detector Pairs used. The vertical lines 1305, mark movement artifacts in the traces. The movement artifacts are large discontinuous changes in the signals throughout the experiment. Trace 1300₅ represents the calculated attenuation (absorption) determined by adding/subtracting the attenuations from the four Source-Detector Pairs. Traces 1300₂ and 1300₃ were added. Traces 1300₁ and 1300₄ were subtracted by the sum of Traces 1300₂ and 1300₃.

As can be seen in Trace 1300₅, the motion artifacts were strongly reduced. Additionally, there is an increase in signal to noise. The signal to noise for Trace 1300₅ is approximately 2.07 times higher than signals to nose of the individual traces 1300₁₋₄.

Various aspects of the present disclosure may be embodied as a program, software, or computer instructions embodied or stored in a computer or machine usable or readable medium, or a group of media which causes the computer or machine to perform the steps of the method when executed on the computer, processor, and/or machine. A program storage device readable by a machine, e.g., a computer readable medium, tangibly embodying a program of instructions executable by the machine to perform various functionalities and methods described in the present disclosure is also provided, e.g., a computer program product.

The computer readable medium could be a computer readable storage device or a computer readable signal medium. A computer readable storage device, may be, for example, a magnetic, optical, electronic, electromagnetic, infrared, or semiconductor system, apparatus, or device, or any suitable combination of the foregoing; however, the computer readable storage device is not limited to these examples except a computer readable storage device excludes computer readable signal medium. Additional examples of the computer readable storage device can include: a portable computer diskette, a hard disk, a magnetic storage device, a portable compact disc read-only memory (CD-ROM), a random access memory (RAM), a read-only memory (ROM), an erasable programmable read-only memory (EPROM or Flash memory), an optical storage device, or any appropriate combination of the foregoing; however, the computer readable storage device is also not limited to these examples. Any tangible medium that can contain, or store, a program for use by or in connection with an instruction execution system, apparatus, or device could be a computer readable storage device.

A computer readable signal medium may include a propagated data signal with computer readable program code embodied therein, such as, but not limited to, in baseband or as part of a carrier wave. A propagated signal may take any of a plurality of forms, including, but not limited to, electro-magnetic, optical, or any suitable combination thereof. A computer readable signal medium may be any computer readable medium (exclusive of computer readable storage device) that can communicate, propagate, or transport a program for use by or in connection with a system, apparatus, or device. Program code embodied on a computer readable signal medium may be transmitted using any appropriate medium, including but not limited to wireless, wired, optical fiber cable, RF, etc., or any suitable combination of the foregoing.

The term “fNIRS controller” as may be used in the present disclosure may include a variety of combinations of fixed and/or portable computer hardware, software, peripherals, and storage devices. The “fNIRS controller” may include a plurality of individual components that are networked or otherwise linked to perform collaboratively, or may include one or more stand-alone components. The hardware and software components of the “fNIRS controller” of the present disclosure may include and may be included within fixed and portable devices such as desktop, laptop, and/or server, and network of servers (cloud).

The terminology used herein is for the purpose of describing particular embodiments only and is not intended to be limiting the scope of the disclosure and is not intended to be exhaustive. Many modifications and variations will be apparent to those of ordinary skill in the art without departing from the scope and spirit of the disclosure.

What is claimed is:

1. A Functional Near-Infrared Spectroscopy (fNIRS) system comprising at least four sensor assemblies, each sensor assembly having at least one optode system including a light source and a light detector, the light source emitting light at a preset duty cycle, each of the at least four sensor assemblies being spaced from one another, the light source being configured to emit light, the light detector in a sensor assembly is capable of detecting emitted light from the light sources from other sensor assemblies, wherein a light source in one sensor assembly and a light detector in another sensor assembly forms a source-detector pair, the light detector in the source-detector pair receives emitted light from the light source in the source-detector pair, the at least four sensor assemblies forming at least twelve source-detector pairs, the light detectors producing at least twelve signals, respectively representing the detected light detected by the light detector in the source-detector pair; and
a controller electrically coupled to each of the at least four sensor assemblies, the controller configured to:
control the light source in each of the at least four sensor assemblies,
receive respective signals from each light detector,
select at least four signals of the at least twelve signals for collective processing, and
process the selected at least four signals of the at least twelve signals to determine absorption, the at least four signals being received from source-detector pairs forming a closed loop arrangement.

2. The fNIRS system of claim 1, further comprising:
a head cap, wherein at least a portion of the at least four sensor assemblies being inserted in mounting slots in the head cap.

3. The fNIRS system of claim 1, wherein the absorption is determined based on values of the received signals and a preset power of the emitted light source.

4. The fNIRS system of claim 1, wherein four sensor assemblies of the at least four sensor assemblies form a square configuration.

5. The fNIRS system of claim 4, wherein the four sensor assemblies forming the square configuration comprise a first sensor assembly, a second sensor assembly, a third sensor assembly and a fourth sensor assembly, the first sensor assembly being adjacent to the second sensor assembly and fourth sensor assembly, the third sensor assembly being adjacent to the second sensor assembly and four sensor assembly, the first sensor assembly being across from the third sensor assembly and the second sensor assembly being across from the fourth sensor assembly, wherein respective light sources and light detectors in the first sensor assembly and the third sensor assembly form source-detector pairs, respective light sources and light detectors in the second sensor assembly and fourth sensor assembly form source-detector pairs,
the controller being further configured to add the values of the respective signals received from light detectors of source-detector pairs of the first sensor assembly and third sensor assembly and signals received from light detectors of the second sensor assembly and the fourth sensor assembly and subtract the value of signals received from light detectors other source-detector pairs to determine absorption.

6. The fNIRS system of claim 5, wherein the selected at least four signals from the at least twelve signals includes a signal from the light detector in the source-detector pair of the first sensor assembly and the third sensor assembly, a signal from the light detector in the source-detector pair of the second sensor assembly and the fourth sensor assembly, a signal from the light detector in a source-detector pair of the second sensor assembly and the third sensor assembly and a signal from the light detector in a source-detector pair of the first sensor assembly and the fourth sensor assembly.

7. The fNIRS system of claim 6, wherein the value of the signal from the light detector in the source-detector pair of the first sensor assembly and the third sensor assembly is added to the value of the signal from the light detector in the source-detector pair of the second sensor assembly and the fourth sensor assembly and wherein the value of the signal from the light detector in a source-detector pair of the second sensor assembly and the third sensor assembly is subtracted from the added signals and the value of the signal from the light detector in a source-detector pair of the first sensor assembly and the fourth sensor assembly is subtracted from the added signals.

8. The fNIRS system of claim 1, wherein the controller sequentially controls the light source in each of the at least fourth sensor assemblies to emit light.

9. The fNIRS system of claim 1, wherein the controller controls the light source in each of the at least fourth sensor assemblies to simultaneously emit light.

10. The fNIRS system of claim 9, wherein the emitted light from the light source in each of the at least fourth sensor assemblies includes an embedded unique high frequency signal.

11. The fNIRS sensing system of claim 1, wherein each sensor assembly has a first optode system and a second optode system separated by a first distance, wherein each sensor assembly is separated from another sensor assembly by a second distance, wherein the second distance is greater than the first distance, the first optode system including a light source, and the second optode system including light detector.

12. A sensor assembly comprising:
a tubular member, the tubular member having a distal portion and a proximal portion, the tubular member having at least one axial opening extending the length of the tubular member from the proximal portion to the distal portion forming a shaft configured to receive at least a portion of an optode, the at least one axial opening having a diameter of a preset size, the tubular member having an external surface;
a first support and a second support mounted to the external surface of the tubular member;
an urging member support opposing a proximal facing portion of the tubular member when the optode is inserted in the shaft;
a first set of urging members including a first urging member and a second urging member, the first urging member extending between the urging member support and the first support, the second urging member extending between the urging member support and the second support, the first and second urging members being configured to, when the optode is inserted in the shaft, urge the optode to a target area; and
a motor configured to, when the optode is inserted in the shaft, vibrate the optode at a first frequency.

13. The sensor assembly of claim 12, wherein the tubular member is dimensioned to fit within an insertion space of a standard 10-20 international EEG cap.

14. The sensor assembly of claim 12, wherein the preset size is substantially equal to a diameter of the optode.

15. The sensor assembly of claim 12, further comprising a cap mounted to the distal portion.

16. The sensor assembly of claim 12, further comprising a detachable cap configured to be mounted to the distal portion of the tubular member when the optode is placed within the shaft.

17. The sensor assembly of claim 16, wherein the detachable cap is transparent.

18. The sensor assembly of claim 12, wherein the at least one axial opening is two axial openings, each of the two axial openings extend the length of the tubular member from the proximal portion to the distal portion forming shafts configured to receive at least a portion of a respective optode, the two axial openings having a diameter of a preset size, and wherein the sensor assembly further comprises:
a second urging member support opposing a proximal facing portion of the tubular member when the optode is inserted in a respective shaft;
a second set of urging members, the second set of urging members including a first urging member and a second urging member, the first urging member extending between the second urging member support and the first support mounted to the external surface of the tubular member, the second urging member extending between the second urging member support and the second support, the first and second urging members being configured to, when the respective optode is inserted in the shaft, urge the respective optode to a target area; and
a second motor configured to, when the respective optode is inserted in the shaft, vibrated the respective optode at a first frequency.

19. The sensor assembly of claim 12, wherein the at least one axial opening is two axial openings, each of the two axial openings extend the length of the tubular member from the proximal portion to the distal portion forming shafts configured to receive at least a portion of a respective optode, the two axial openings having a diameter of a preset size, and wherein the sensor assembly further comprises:
an second urging member support opposing a proximal facing portion of the tubular member when the optode is inserted in a respective shaft;
a third support and a fourth support mounted to the external surface of the tubular member;
a second set of urging members, the second set of urging members including a first urging member and a second urging member, the first urging member extending between the second urging member support and the third support mounted to the external surface of the tubular member, the second urging member extending between the second urging member support and the fourth support, the first and second urging members being configured to, when the respective optode is inserted in the shaft, urge the respective optode to a target area; and
a second motor configured to, when the respective optode is inserted in the shaft, vibrated the respective optode at a first frequency.

20. The sensor assembly of claim 12, wherein the urge member support includes an opening, the opening being configured to receive the optode.

21. The sensor assembly of claim 12, wherein the first frequency is determined based on a data sampling rate.
